(19) United States
(12) Patent Application Publication
(10) Pub. No.: US 2017/0156396 A1
(43) Pub. Date: Jun. 8, 2017

(54) HOMEOPATHIC AGENT ALLOWING SMOKERS TO QUIT SMOKING

(71) Applicant: Marianna GROSS, Illingen (DE)

(72) Inventor: Marianna GROSS, Illingen (DE)

(21) Appl. No.: 15/325,460

(22) PCT Filed: Jul. 10, 2015
(86) PCT No.: PCT/EP2015/065847

§ 371 Date: Feb. 9, 2017

(30) Foreign Application Priority Data
Jul. 11, 2014 (DE) 20 2014 103 194.4

(51) Int. Cl. A24F 47/00; A61K 36/81

(52) U.S. Cl. CPC A24F 47/00; A61K 36/81; A24B 15/28

(57) ABSTRACT

The present invention relates to a homeopathic agent allowing smokers to quit smoking, method for its production and its use as a medicament in smoking cessation therapy. The agent allowing smokers to quit smoking is characterized in that it is a homeopathic agent and contains tobacco products or the ashes of tobacco products in homeopathic dilutions.

### FIELD OF THE INVENTION

The invention relates to a homeopathic agent allowing smokers to quit smoking. The agent contains tobacco products or the ash of tobacco products, such as, for instance, cigarettes or cigars, etc., in homeopathic dilutions. The invention furthermore relates to the use of the agent for a medicament for smoking cessation therapy.

### BACKGROUND OF THE INVENTION

It is known that smoking quickly leads to signs of addiction. Many smokers who would like to quit smoking are thus faced with withdrawal problems which lead to the smoker relapsing in many cases. Nicotine plasters are available on the market, which allow the absorption of nicotine via the skin. However, these plasters do not solve the problem of nicotine dependency, nor do they offer a replacement for the process of smoking, and thus smokers often fail to permanently quit smoking even if nicotine plasters are used.

Additionally, homeopathic agents allowing smokers to quit smoking are also known. DE 20 2008 007 923 U1 describes a suppository that contains at least Nicotiana tabacum. US 2006/062835 A1 describes a nicotine withdrawal method, in which in a first phase a pressure pad is applied to the patient's ear, in a second phase the patient then orally ingests a withdrawal agent, in a third phase, which overlaps with the first phase, the patient orally ingests a stress-reducing substance, and in a fourth phase these treatment steps are continued. US 2005/100513 A1 describes a nicotine withdrawal method, in which two homeopathic substances are administered simultaneously, the first substance counteracting the nicotine addiction and the second substance serving to detoxify the body. Finally, RU 2 143 274 C1 describes a homeopathic agent allowing smokers to quit smoking, which is administered in the form of sugar granules. A further agent allowing smokers to quit smoking, which is based on a mixture of different active substances that are present in homeopathic dilutions, is known from EP 2 070 544 B1.

### DESCRIPTION OF THE INVENTION

The object of the invention is to provide an agent allowing smokers to quit smoking, with which it is possible for smokers to permanently quit smoking in the majority of cases. This object is solved according to the invention by the agent according to claim 1. The agent is configured as a homeopathic agent and contains tobacco products or the ash of tobacco products in homeopathic dilutions.

Tobacco products within the meaning of the present invention are cigarettes, including tobacco cigarettes and cannabis cigarettes, cigars, cigarette tobacco and pipe tobacco as well as finished tobacco for use in the cited products, without the tobacco being processed beforehand into cigarettes or cigars or other tobacco products, such as, for example, cigarette or cigar tobacco.

In a first embodiment, the agent contains cigarettes, including tobacco cigarettes and cannabis cigarettes, cigars or pipe tobacco or combinations thereof in homeopathic dilutions. The cigarettes, cigars and/or pipe tobacco are thereby used as a whole, i.e. including the paper and the filter if these are present, and are processed into the desired homeopathic dilutions according to the rules of the German Homeopathic Pharmacopoeia (HAB). Furthermore, finished tobacco for use in tobacco products can be used as such, without the tobacco having been processed beforehand into cigarettes or cigars or other tobacco products.

In a second embodiment, the agent contains the ash of cigarettes, cigars or pipe tobacco or combinations thereof in homeopathic dilutions. The respective ashes are processed into the desired homeopathic dilutions according to the rules of the German Homeopathic Pharmacopoeia.

The ash of cigarettes can include the ash of tobacco cigarettes or the ash of cannabis cigarettes or combinations thereof.

The ash that is used for the agent allowing smokers to stop smoking as according to the invention is the ash of burned cigarettes, including the cigarette paper, of burned cigars or of burned pipe tobacco, such as results when the respective products are smoked.

Furthermore, the ash that results when burning finished tobacco for use in tobacco products can be used as such, without the tobacco having been processed beforehand into cigarettes or cigars or other tobacco products.

In a further embodiment, the ash of burned cigarettes, including the cigarette paper and a possibly present cigarette filter, or the ash of burned cigars can be used, which results from the complete burning of a cigarette or a cigar for instance over the flame of a Bunsen burner.

Each brand of cigarette, cigar, cigarette tobacco or pipe tobacco contains different varieties of tobacco, mixtures of tobacco varieties and additives such as, for example, addiction intensifiers, fragrances, preservatives, etc. For personalized smoking cessation therapy, it is therefore also possible to use the ash of a specific brand of cigarette, cigar, cigarette tobacco or pipe tobacco for the agent allowing smokers to quit smoking as according to the invention.

The agents allowing smokers to quit smoking as according to the invention contain the ash of tobacco products in homeopathic dilutions, whereby the respective ash can be used in D, C and Q potencies. Preferred potencies are D1 to C1000. Particularly preferred potencies are D6, D8, D12 and D30.

The cited potencies correspond to the commonly used and known potencies of homeopathic preparations and are produced according to the provisions of the German Homeopathic Pharmacopoeia. The letter in combination with the respective stage of potency thus indicates the degree of dilution of the ash in the agents allowing smokers to quit smoking as according to the invention. For example, the potency D30 stands for a dilution of 1:10³⁰.

The production of the agent allowing smokers to quit smoking as according to the invention follows the rules and provisions of the respectively valid German Homeopathic Pharmacopeia. According thereto
a) a mother tincture is produced from the tobacco products as such, i.e. from the respective cigarettes, cigars, cigarette or cigar tobacco as such or from pipe tobacco, and is subsequently homeopathically potentized, or
b) a trituration is produced from the ash of the tobacco products which results when the respective products are smoked or completely burned, and is subsequently homeopathically potentized.

For example, the D6 dilution of an aqueous solution of the cigarettes of a specific brand as such can be produced in the following manner according to the German Homeopathic Pharmacopeia (HAB).

### Mother Tincture:

In accordance with the methods of maceration or percolation as described in the section Tinctures of the monograph on Extracts in the European Pharmacopeia (Ph. Eur.), a mother tincture is produced from 1 part of fresh or dried cigarettes of a specific brand and 10 parts of ethanol of a suitable concentration.

### Potentization:

The mother tincture corresponds to the 1^{st} decimal dilution D1. The 2^{nd} decimal dilution (D2) is produced from 1 part of the mother tincture and 9 parts of water for injection. The same procedure is carried out for subsequent dilutions to arrive at the 3^{rd}, 4^{th}, 5^{th} and finally the 6^{th} decimal dilution D6.

The D6 dilution of an aqueous preparation of the ash of cigarettes of a specific brand can be produced as follows according to the German Homeopathic Pharmacopeia (HAB).

In the first step, a trituration of the ash is produced with lactose monohydrate as the carrier. For this purpose, the ash is reduced in size to a suitable degree of reduction and is then mechanically triturated with lactose monohydrate in a ratio of 1 to 10 (1^{st} decimal dilution D1), the triturating machine comprising a scraper that ensures uniform trituration. Initially, one third of the lactose monohydrate used as the carrier is thereby triturated. The ash is then added, triturated and finally the rest of the lactose monohydrate is added and triturated in two equal portions. The operating time for the machine production of the trituration is at least one hour.

The same procedure is carried out for the following dilution steps up to the 4^{th} decimal dilution D4.

In order to produce the liquid dilution D6, 1 part of the trituration D4 is dissolved in 9 parts of water for injection and then shaken. The liquid dilution D6 is produced, by shaking, from 1 part of this dilution with 9 parts of water for injection.

The dilutions obtained in such a manner can then be processed individually or in combination into suitable dosage forms.

The agents allowing smokers to quit smoking as according to the invention can be provided in all common dosage forms for administering homeopathic medicaments. Suitable dosage forms are drops, globules, a sugar solution in liquid or solid form, chewing gums, creams, ointments, gels, skin rollers, suppositories or an aqueous solution, whereby the aqueous solution can be configured for injection, inhalation, spraying or vaporization.

The aqueous solution can be an isotonic NaCl solution. The aqueous solution can also be an alcoholic solution.

The agents allowing smokers to quit smoking as according to the invention are preferably in the form of aqueous solutions for injection, drops and globules. The D6 dilutions as described above can, for example, be transformed into an aqueous solution for injection.

It is particularly preferred for the agent allowing smokers to quit smoking as according to the invention to be an aqueous solution for injection. In this case, the diluent is water and it is an aqueous solution of the active substances. With a single injection of 2 ml of this agent into the earlobe, it is possible to achieve immediate cessation of smoking with a high degree of success. Surprisingly, no withdrawal symptoms generally occur even in the first 24 hours and, in the majority of cases, long-term cessation of smoking is achieved. In rare cases, it is necessary to administer a second injection in order to achieve complete cessation of smoking.

In a further embodiment, the invention relates to the use of the agent allowing smokers to quit smoking as according to the invention for a medicament for smoking cessation therapy, or to the agent allowing smokers to quit smoking as according to the invention for use as a medicament for smoking cessation therapy.

In a particularly preferred embodiment of this use, the agent for allowing smokers to quit smoking can contain cigarettes, cigars, cigarette tobacco and/or pipe tobacco or the respective ash thereof in homeopathic dilutions of the brand which the respective patient smoked prior to the start of the smoking cessation therapy.

1-8. (canceled)

9. A homeopathic agent allowing smokers to quit smoking, characterized in that the agent contains cigarettes, including paper and filter if these are present, in homeopathic dilutions.

10. The homeopathic agent allowing smokers to quit smoking according to claim 9, characterized in that the agent contains cigarettes in the homeopathic dilutions D1 to C1000.

11. The homeopathic agent allowing smokers to quit smoking according to claim 9, characterized in that the agent contains cigarettes in the homeopathic dilutions D6, D8, D12 or D30.

12. The homeopathic agent allowing smokers to quit smoking according to claim 10, characterized in that the agent contains cigarettes in the homeopathic dilutions D6, D8, D12 or D30.

13. The homeopathic agent allowing smokers to quit smoking according to claim 9, characterized in that the homeopathic agent is in the form of drops, globules, a sugar solution in liquid or solid form, chewing gums, creams, ointments, gels, skin rollers, suppositories or an aqueous solution, the aqueous solution being configured for injection, inhalation, spraying or vaporization.

14. The homeopathic agent allowing smokers to quit smoking according to claim 10, characterized in that the homeopathic agent is in the form of drops, globules, a sugar solution in liquid or solid form, chewing gums, creams, ointments, gels, skin rollers, suppositories or an aqueous solution, the aqueous solution being configured for injection, inhalation, spraying or vaporization.

15. The homeopathic agent allowing smokers to quit smoking according to claim 12, characterized in that the homeopathic agent is in the form of drops, globules, a sugar solution in liquid or solid form, chewing gums, creams, ointments, gels, skin rollers, suppositories or an aqueous solution, the aqueous solution being configured for injection, inhalation, spraying or vaporization.

16. The homeopathic agent allowing smokers to quit smoking according to claim 13, characterized in that the aqueous solution is an isotonic NaCl solution.

17. The homeopathic agent allowing smokers to quit smoking according to claim 14, characterized in that the aqueous solution is an isotonic NaCl solution.

18. The homeopathic agent allowing smokers to quit smoking according to claim 15, characterized in that the aqueous solution is an isotonic NaCl solution.

19. A method for producing the homeopathic agent allowing smokers to quit smoking according to claim 9, characterized in that the production method is a homeopathic production method comprising the production of a mother tincture from the cigarettes, which is subsequently homeopathically potentized.

20. A method for producing the homeopathic agent allowing smokers to quit smoking according to claim 13, characterized in that the production method is a homeopathic production method comprising the production of a mother tincture from the cigarettes, which is subsequently homeopathically potentized.

21. A method for producing the homeopathic agent allowing smokers to quit smoking according to claim 16, characterized in that the production method is a homeopathic production method comprising the production of a mother tincture from the cigarettes, which is subsequently homeopathically potentized.

22. A method for producing the homeopathic agent allowing smokers to quit smoking according to claim 18, characterized in that the production method is a homeopathic production method comprising the production of a mother tincture from the cigarettes, which is subsequently homeopathically potentized.

23. The homeopathic agent allowing smokers to quit smoking according to claim 9 for use as a medicament for smoking cessation therapy.

24. The homeopathic agent allowing smokers to quit smoking according to claim 10 for use as a medicament for smoking cessation therapy.

25. The homeopathic agent allowing smokers to quit smoking according to claim 18 for use as a medicament for smoking cessation therapy.

26. The homeopathic agent allowing smokers to quit smoking for the use according to claim 23, characterized in that the agent allowing smokers to quit smoking contains cigarettes in homeopathic dilutions of the brand which the respective patient smoked prior to the start of the smoking cessation therapy.

27. The homeopathic agent allowing smokers to quit smoking for the use according to claim 24, characterized in that the agent allowing smokers to quit smoking contains cigarettes in homeopathic dilutions of the brand which the respective patient smoked prior to the start of the smoking cessation therapy.

28. The homeopathic agent allowing smokers to quit smoking for the use according to claim 25, characterized in that the agent allowing smokers to quit smoking contains cigarettes in homeopathic dilutions of the brand which the respective patient smoked prior to the start of the smoking cessation therapy.
